(19) United States
(12) Patent Application Publication
(10) Pub. No.: US 2006/0264746 A1
(43) Pub. Date: Nov. 23, 2006

(54) Diagnostic imaging system control with multiple control functions

(75) Inventors: Janice Frisa, Bothell, WA (US); McKee Dunn Poland, Bothell, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 10/557,341

(22) PCT Filed: May 6, 2004
(86) PCT No.: PCT/IB04/01707

§ 371 Date: Nov. 21, 2005

Related U.S. Application Data
(60) Provisional application No. 60/473,855, filed on May 27, 2003.

(51) Int. Cl. A61B 8/00

(52) U.S. Cl. 600/437; 600/447

(57) ABSTRACT

A user control of a medical imaging system is normally operated by the user to control a function of the system such as the position of one image plane relative to another. There are a number of modes in which the relative plane position can be varied, such as varying the relative elevation position or the relative lateral position of the planes. To switch from one mode to the other the user moves the user control rapidly to effect a change of the mode of operation being controlled. A processor senses the high speed motion of the user control and changes the mode being controlled by the user control to a new mode. The user control may comprise a trackball, rotary knob, linear slider, touchpad, or other control panel user control.

This invention relates to diagnostic imaging systems, and more particularly, to controls for a diagnostic imaging system which perform multiple functions.

Diagnostic imaging systems, such as diagnostic ultrasound imaging systems, are complex instruments with a multitude of different control parameters. The user can vary numerous setting in order to obtain the best diagnostic image. For example, the user can adjust the transmit power, receiver gain, time gain control, lateral gain variation, image depth, and imaging modes, among other things. Because there are so many variable control parameters, fully featured imaging systems have control panels with a significant number of controls. Accordingly, designers of such systems are constantly trying to provide control panels with simpler control configurations, while retaining the ability to easily and intuitively control all imaging variables. In particular, such control panels usually try to arrange the control configuration so that the controls needed for a particular procedure are closely grouped on the control panel so that the user can reach them quickly, preferably without moving the hand. For example, the main controls on a diagnostic imaging system control panel are the trackball and the keys which are arrayed in an arc around the trackball. The trackball is used primarily to move cursors and other markers around the display screen. The keys arrayed around the trackball are those most often used by the clinician, such as an “image freeze” key, a “print” key, a “measurement” key, a “select” key and an “update” key. However the space available for such keys is limited. It would be desirable for a control panel to have a control configuration that enables the control of greater functionality within easy reach of the user but without undue mechanical and control complexity.

In accordance with the principles of the present invention, a variable control for a diagnostic imaging system is provided which performs multiple functions. In one embodiment the control is one with a continuous range of control such as a trackball, slider, or knob. When the control is manipulated in a normal manner, it provides a first type of control function. When the control is manipulated in a rapid fashion, the velocity of the control action is sensed and the control changes to a different control function. An embodiment of this invention can obviate the need for a separate control, or for additional hand motion of the user, in order to control multiple functions.

In the drawings:

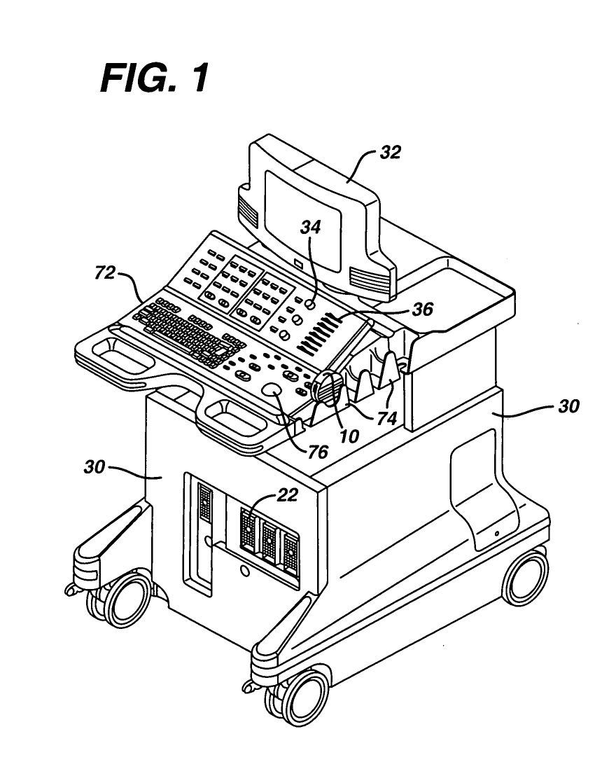

FIG. 1 illustrates a diagnostic ultrasound system constructed in accordance with the principles of the present invention;

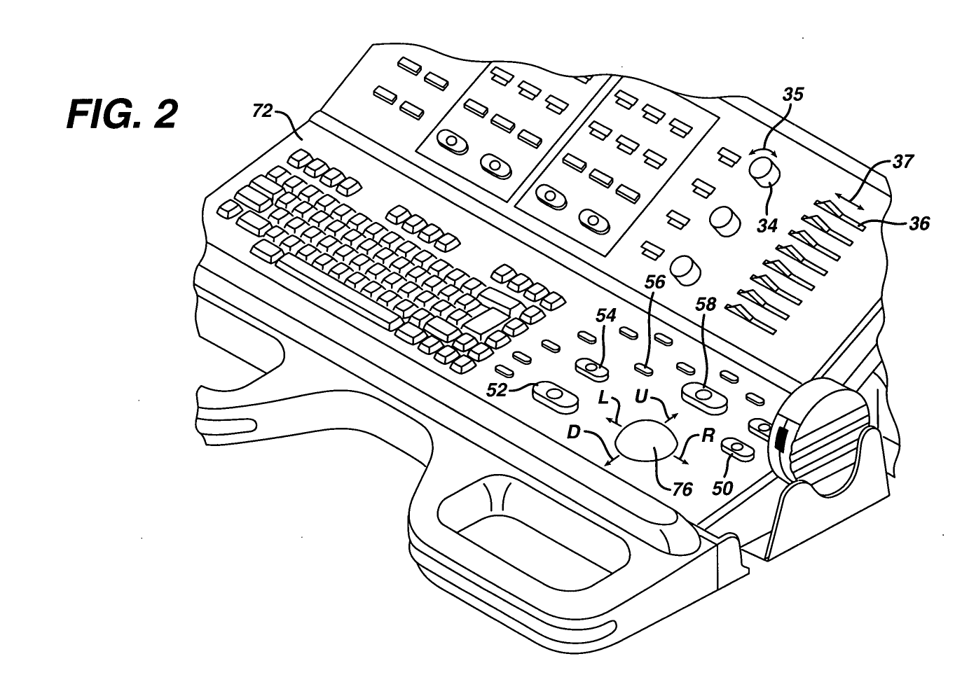

FIG. 2 is a close-up view of a portion of the control panel of the ultrasound system of FIG. 1;

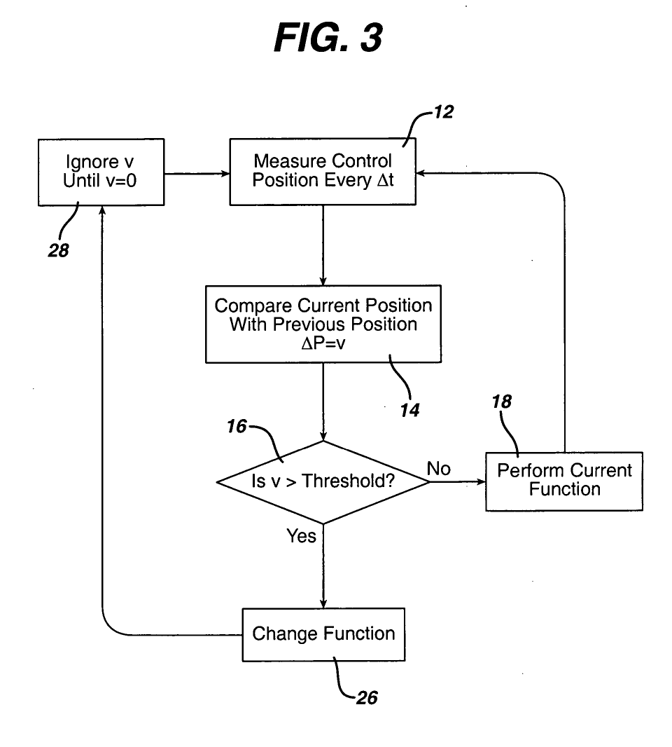

FIG. 3 is a flowchart diagramming the operation of a control in accordance with the principles of the present invention; and

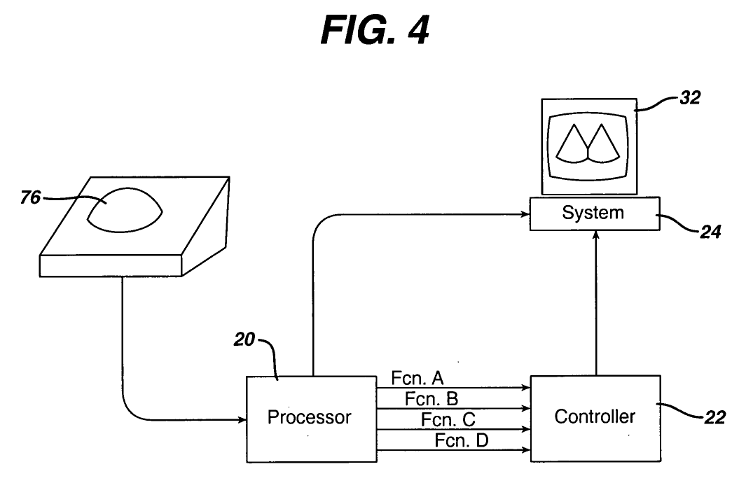

FIG. 4 illustrates an instrumentation system in which a control can be used to change to any of four different functions.

Referring first to FIG. 1, a cart-borne diagnostic ultrasound system constructed in accordance with the principles of the present invention is shown in a perspective view. The lower portion 30 of the system encloses a power supply and circuit boards by which the system processes and displays ultrasound signals. A number of connectors are shown on the front of this lower portion 30, including a connector 22 for the cable 20 of an ultrasound probe 10. The probe 10 can be stored in a probe holder 74 when not in use. Located on top of the lower portion 30 is a control panel 72 containing the system controls by which a user operates and controls the ultrasound system. Above the control panel 72 is a display 32 on which the ultrasound images produced by the system 30 are displayed.

The control panel 72 includes a number of controls which have a continuous range of adjustment. Three such controls are a trackball 76, a slider 36, and a knob 34. These controls have a continuous range of motion through which the user moves the control when using the control to adjust a system function or parameter. The trackball 76, for instance, can be rolled or spun by the operator in a continuous manner. The slider 36 and knob 34 will generally not have the unlimited continuous range of motion of the usual trackball, but will generally have ranges of travel which are mechanically limited. The travel of the slider 36 is limited by the length of the control and its opening on the control panel, for example. The knob 34 will generally have end stops at its minimum and maximum limits of rotation. However, knobs which can be continuously turned are also known and can also be employed in an embodiment of the present invention.

FIG. 2 is a closeup view of a portion of the control panel 72 which contains a trackball 76, a slider 36, and a knob 34. The trackball on an ultrasound system generally performs the functions described above. Sliders 36 are often employed to set the breakpoints of the system's time gain compensation curve. Knobs are used for functions such as gain or power adjustment. As shown in FIG. 2, a knob 35 will have a rotational range of control adjustment as indicated by arcuate arrow 35. A slider 36 will have a linear range of control adjustment as indicated by the linear arrow 37. The trackball 76 can generally be rotated in any direction including the four illustrated directions of Up, Down, Left and Right as the arrows indicate. These controls differ from a control with discrete settings such as a pushbutton which has only two positional settings. Arrayed around the trackball 76, as discussed above, are control keys or pushbuttons 50-58 which control such functions as freezing the image on the display, printing the displayed image, measuring a designated part of the anatomy on the display, selecting a setting designated by the display cursor, or updating the display.

In accordance with the principles of the present invention, a control with a continuous range of adjustment such as those described above is used to control several functions. For example, the trackball 76 can be used to control the location of an image plane in a 3D ultrasound imaging mode known as “biplane.” The biplane mode is described in detail in US patent [application serial number 10/231,704}, entitled “BIPLANE ULTRASONIC IMAGING” and U.S. Patent (attorney docket ATL-326, filed May 12, 2003), entitled “IMAGE ORIENTATION DISPLAY FOR A THREE DIMENSIONAL ULTRASONIC IMAGING SYSTEM” which are incorporated herein by reference. In the biplane mode described in these patents, an array transducer images one image plane which extends normal to the face of the transducer which is referred to as the reference image. The reference image is oriented with respect to the transducer as would a normal 2D image during two dimensional imaging. In the biplane mode there is a second image, the plane of which can be moved with respect to the reference image. In one biplane function the reference image and the second image have a common center line. The second image can be rotated about this common center line. This means that the two images can be co-planar, can be oriented at 90° with respect to each other, or any intermediate rotational angular orientation.. This is known as the “rotate” biplane mode. Another biplane function is known as the “lateral tilt” biplane mode. In this mode the two images are oriented at 90° with respect to each other, with the center line of the second line sharing a selectable line of the reference image. When the two images are sector images, this means that the second image can be tilted away from a normal orientation to the probe, from one lateral side of the reference image to the other. Yet a third biplane function is the “elevation tilt” biplane mode, in which the second image can be moved in the elevation direction of the reference image. This means that the two images can be co-planar, or that the second image can move to an elevation plane in front of or behind the plane of the reference image.

In accordance with the principles of the present invention, the trackball 76 is used to control the position of the second plane of biplane mode. The trackball can also be used to switch to one or more of the other biplane functions. This is done by rapidly spinning the trackball. When the ultrasound system senses the high velocity movement of the trackball, the system switches to another biplane function. The trackball is then used to control the position of the second plane in the new biplane function. This enables the user to move the second plane in one biplane function to search for the best view of anatomy of interest. If the user is dissatisfied with these views, he can momentarily spin the trackball to switch to a new biplane function, in which the second plane can be moved to a different range of orientations with respect to the reference plane. In this way the clinician can maintain one anatomy in view in the reference image to establish a reference view, and can manipulate the second plane to a wide range of other views and orientations, all with the same user control.

For example, suppose that the clinician is holding the ultrasound probe so that a valve of the heart is shown in the plane of the reference image. The clinician is operating elevation tilt function of the biplane mode. As the clinician gently moves the trackball with his fingers, the second image moves from being co-planar with the reference plane to positions which intersect the heart chamber in front of and substantially parallel to the heart valve. If the heart valve has a jet, the second plane ay cut across this jet. But in this orientation the jet will generally appear only as a pot in the elevation plane of the second image. The clinician may now want to see the full length of the jet in the second image plane, which extends approximately normal to the valve. This may require switching to the lateral tilt function of the biplane mode. To make this change the clinician gives the trackball a quick, rapid spin with the fingers. The ultrasound system senses this high velocity movement of the trackball and switches to the lateral tilt function. The clinician can now resume normal control of the trackball to laterally tilt the second plane until the entire length of the jet is captured in the plane of the second image.

FIG. 3 illustrates a flowchart of a procedure by which the ultrasound system can sense the high velocity motion of the trackball or other user control. A trackball, for example, can have two or four potentiometers or quadrature shaft encoders which are adjusted by the rolling of the trackball. One shaft encoder is adjusted and produces output signals when the trackball is moved up or down, and another shaft encoder is adjusted and produces output signals when the trackball is moved left or right. The direction in which the adjustment is made is indicated by a direction bit (+ or −) which distinguishes up from down and left from right. Alternatively there can be a shaft encoder for each of the four directions. When the trackball is moved in directions other than these four orthogonal directions several shaft encoders are adjusted simultaneously, and their combined movements can be resolved into the direction of the trackball's motion. In this way a cursor can be moved from one point on a display screen in a straight line to any other point on the screen, for instance.

In box 12 of FIG. 3 the control position P is measured every Δt time increment. For example, the current values of the trackball's shaft encoders can be read by a processor in the ultrasound system every 50 milliseconds. Each time the control position is measured the current position is compared with the previously measured position (box 14). This comparison is a value representing a change in position ΔP which, since the change occurred in a time interval Δt, is equal to a velocity v. If the consecutively measured positions of the control are the same, this means that the control has not been moved, and ΔP=v=0. But if there is a change of position in the 50 millisecond interval, the control has moved in position ΔP in an interval Δt=a non-zero v.

In box 16 v is compared to a threshold. The threshold determines whether the control's motion is normal low velocity motion, in which case no functional change is made, or that the control is being moved at a high velocity which dictates a change in the function of the control. In one embodiment the threshold may be set at the factory by the ultrasound system manufacturer in consideration of empirical knowledge of how the control is manipulated in normal operation. In another embodiment the threshold may be adjusted by the user in consideration of his or her personal style of control manipulation. In the second embodiment the user will set the threshold so that all of the velocities of his normal usage of the control are below the threshold. This prevents the control from inadvertently switching to a different function during normal control adjustment. If v is below the threshold, the control continues to perform its current function (box 18) and the motion of the control is used accordingly.

For example, if the control was being used to adjust the elevation tilt of the second plane, the new setting of the trackball is used to effect a new elevation tilt position of the second plane.

However, if v is above the threshold, the control has been moved at an abnormally high velocity. The function of the control is then changed by the ultrasound system (box 26). The control may continue to move at the abnormally high velocity for several or many Δt measurement intervals. During this period the v value is ignored for control purposes until the control resumes a normal velocity v or v=0 (box 28). When the control is operating normally again or has come to rest, the sequence continues (box 12, etc.) The use of the high velocity to change the function of the control may be used to toggle back and forth between two different functions. In the above example a quick movement of the trackball 76 while in the elevation tilt mode can switch to the lateral tilt mode. When the user is in the lateral tilt mode a quick spin of the trackball can return the control to operation of the elevation tilt mode. Alternatively, rapid movement of the trackball can switch the control to the rotate biplane mode, from which another rapid movement switches to the elevation tilt mode.

In another embodiment the direction of the control movement can dictate the new function. As FIG. 2 shows, the trackball 76 has four distinct directions of rotation: up, down, left and right. Spinning the trackball upward can cause a change to a first new function while spinning the trackball downward can cause a change to a second new function. Spinning the trackball to the left or to the right can cause a change to a third or fourth new function. Similar operation is possible with the knob 34, where a quick clockwise movement can cause a change to one new function while a quick counterclockwise movement can cause a change to another new function. Similarly, quickly sliding the slider 36 to the left can cause a change to one new function while quickly sliding the slider to the right can cause a change to another new function.

A system which uses the flowchart of FIG. 3 to change a control function is illustrated in block diagram form in FIG. 4. The potentiometer output of a slider or knob control or the shaft encoder outputs of the trackball 76 are coupled to a processor 20. The processor measures the potentiometer or shaft encoder values and hence the position of the ball of the trackball or other control setting on a periodic basis, and compares successive position settings of the potentiometer or shaft encoders to detect velocities in each of four distinct directions. If the velocity of the trackball is within nominal control limits (i.e., relatively low), the trackball position signals are coupled to the imaging system for control of the current function. A high velocity in predominately one of the four distinct directions causes a change to a designated function: Function A, Function B, Function C or Function D. The function signals may be on separate lines as shown in this embodiment or may use one control line or one multi-bit bus. The function signals are applied to a controller 22, which responds to a new function signal to change the function being controlled by the trackball 76. The controller 22 is coupled to the imaging system 24 to effect this change. The function being adjusted by the trackball such as the position of a cursor or image plane is shown on the system display 32.

Other control devices than those shown in FIG. 2 may be used in an implementation of the present invention. For example, the user control may be a capacitive touchpad which is operated by the touch of a user. When the user slides a finger slowly across the touchpad the varying capacitance adjusts the operation of a function in the normal manner, and when the user slides a finger quickly across the touchpad the system being controlled switches to another function which is being controlled by the touchpad. In another embodiment a variable inductance can be controlled by the user control.

In the claims:

1. A medical imaging system which is operable to perform a plurality of different functions and having a user control which can be manipulated by a user to adjust the operation of a plurality of functions comprising:
a user control output signal, produced by the user control in response to manipulation of the user control, which indicates the current setting of the user control;
a processor, responsive to the user control output signal, which detects manipulation of the user control in a first manner in the absence of a second manner and in the first manner simultaneously with the in a second manner; and
a function controller, responsive to the detection of manipulation of the user control in the first manner in the absence of the second manner and in the first manner simultaneously with the second manner, which acts to change different functions being operated by the user control.

2. The medical imaging system of claim 1, wherein the first manner is direction of control movement and wherein the second manner is velocity of control movement.

3. The medical imaging system of claim 2, wherein the user control further comprises a variable impedance device, the impedance of which is adjusted by the user during manipulation of the user control.

4. The medical imaging system of claim 3, wherein the variable impedance device comprises a potentiometer.

5. The medical imaging system of claim 3, wherein the user control comprises one of a variable resistive, variable inductive, or variable capacitive device.

6. The medical imaging system of claim 1, wherein the user control output signal comprises a signal indicating the instantaneous position of a control setting being manipulated by a user.

7. The medical imaging system of claim 1., wherein the processor is responsive to successive user control output signals produced over time to compute one of the manners in which the user control is being manipulated.

8. The medical imaging system of claim 7, wherein the processor is responsive to successive user control output signals produced over time to compute the velocity at which the user control is being manipulated.

9. The medical imaging system of claim 8, wherein the processor is further responsive to successive user control output signals produced over time to compute the direction in which the user control is being manipulated.

10. The medical imaging system of claim 9, wherein the function controller is further responsive to the velocity and direction in which the user control is being manipulated to select one of a plurality of new functions for control by the user control.

11. The medical imaging system of claim 1, wherein the processor further comprises a threshold value used by the processor to determine whether the user control is being manipulated in the second manner.

12. The medical imaging system of claim 11, wherein the threshold value further comprises a user adjustable threshold value.

13. The medical imaging system of claim 1, wherein the user control comprises one of a trackball, a touchpad, a control knob, or a slider control.

14. The medical imaging system of claim 2, wherein the user control further comprises a variable output signal device, the output signal of which is varied during manipulation of the user control.

15. The medical imaging system of claim 14, wherein the variable output signal device comprises a shaft encoder.

16. In a medical imaging system, a method for controlling a function and changing the function being controlled by a user control comprising:
using the user control when operated in a first manner to control a first function;
sensing use of the user control in a second manner;
changing the function being controlled by the user control when simultaneous use of the first and second manner is sensed; and
using the user control when operated in the first manner in the absence of the second manner to control the changed function.

17. The method of claim 16, wherein using the user control when operated in a first manner comprises using the user control when operated at a relatively low velocity of manipulation to control a function of the system.

18. The method of claim 17, wherein sensing the use of the user control comprises sensing relatively high velocity manipulation of the user control in a control direction.

19. The method of claim 18, wherein sensing relatively high velocity manipulation of the user control further comprises measuring the position of the user control at periodic intervals and comparing different position measurements to determine the velocity of manipulation of the user control.

20. The method of claim 18, wherein sensing the use of the user control further comprises sensing the direction of manipulation of the user control.

21. The method of claim 16, wherein using the user control comprises using a trackball, a rotary knob, a linear slider control, or a touchpad.
